(19) United States
(12) Patent Application Publication
(10) Pub. No.: US 2010/0241038 A1
(43) Pub. Date: Sep. 23, 2010

(54) Phototherapy Method for Assisting Transvenous Lead Placement

(75) Inventors: Ryan Edward Sullivan, Yardley, PA (US); Brian Pryor, Newark, DE (US); Sean X. Wang, Wilmington, DE (US)

Correspondence Address: BWT PROPERTY, INC., 19 SHEA WAY, SUITE 301, NEWARK, DE, 19713, US

(73) Assignee: BWT PROPERTY, INC., Newark, DE (US)

(21) Appl. No.: 12/727,279

(22) Filed: Mar. 19, 2010

Related U.S. Application Data
(60) Provisional application No. 61/161,862, filed on Mar. 20, 2009.

(51) Int. Cl. A61B 17/00; A61N 5/06; A61N 5/067

(52) U.S. Cl. 601/15; 607/88; 607/89; 606/129

(57) ABSTRACT

A phototherapy method for assisting the transvenous lead placement for cardiac resynchronization therapy (CRT) and dilation of occluded veins for any implantable lead placement. The phototherapy treatment helps increase blood flow, reduce inflammation, and enhance angiogenesis to facilitate optimal positioning of the leads.

### REFERENCE TO RELATED APPLICATION

This application claims an invention which was disclosed in Provisional Patent Application No. 61/161,862, filed Mar. 20, 2009, entitled “LASER THERAPY METHOD FOR ASSISTING TRANSVENOUS LEAD PLACEMENT”. The benefit under 35 USC §119(e) of the above mentioned United States Provisional Applications is hereby claimed, and the aforementioned application is hereby incorporated herein by reference.

### FIELD OF THE INVENTION

This invention generally relates to a phototherapy method, and more specifically to a phototherapy method for assisting transvenous lead placement.

### BACKGROUND

Cardiac resynchronization therapy (CRT) is a new therapeutic procedure that can relieve congestive heart failure (HF) symptoms by improving the coordination of the heart's contractions. The CRT pacing device has three leads (wires) that are implanted through a vein in the right atrium and right ventricle and into the coronary sinus veinous anatomy to pace the left ventricle. The optimal lead position is in a mid-lateral venous anatomy, which is imperative to provide the best resynchronization. One technical challenge facing the transvenous lead placement is that many heart failure patients suffer from distorted coronary sinus anatomy and narrow, irregular veins as resulted from reduced blood flow, which may cause inability or sub-optimal lead placement.

The present invention discloses a phototherapy method for increasing blood flow, dilating blood vessels, and enhancing angiogenesis of the heart in order to assist transvenous lead placement. This method will help increase the percent of leads with optimal lead position which will in turn increase the number of responders to treatment, thereby reducing hospitalizations and the need for transthorasic surgery for epicardial lead placement.

In addition, the described method may also aid in gaining venous access for angioplasty procedures, where balloons, stents, or other medical devices are employed to widen the diameter of blocked blood vessels in the heart, kidney, leg, and neck of the patient.

### SUMMARY OF THE INVENTION

A phototherapy method for assisting the transvenous lead placement for cardiac resynchronization therapy (CRT) and dilation of occluded veins for any implantable lead placement. The phototherapy treatment helps increase blood flow, reduce inflammation, and enhance angiogenesis to facilitate optimal positioning of the leads.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 shows one exemplary phototherapy system for assisting transvenous lead placement in a patient receiving an implantable cardiac device.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a phototherapy method for assisting transvenous lead placement. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

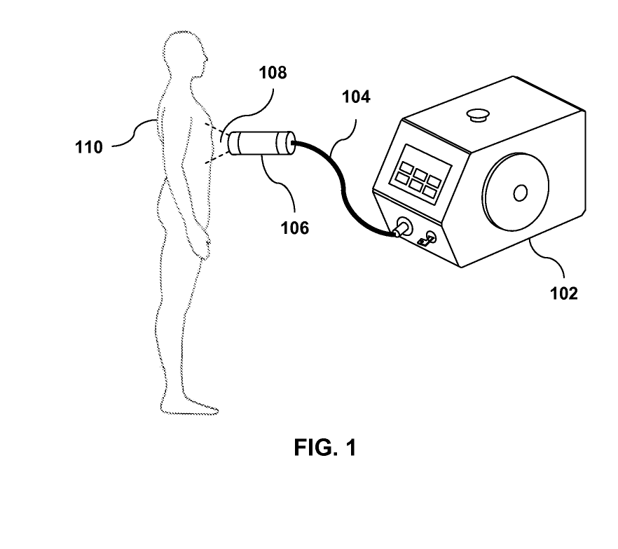

Referring to FIG. 1, an exemplary phototherapy system is disclosed for assisting transvenous lead placement in a patient with congestive heart failure (HF) symptom and/or occluded venous structures. The system comprises a laser light source 102 to produce a laser light as well as an optical waveguide 104, such as an optical fiber or a liquid light guide to deliver the laser light from the laser light source 102 to an output wand 106. The output wand 106 controls the divergence angle of the laser light such that an output laser beam 108 with desired power density is produced. The output laser beam 108 is directed toward the chest area of the patient 110 and penetrates into the patient's chest to non-invasively treat the cardiac veinous anatomy, coronary artery, and the surrounding heart tissue. The therapeutic laser light treats the blood vessel and the heart tissue through photothermal and photochemical reactions, e.g. regulation of adenosine triphosphate (ATP) and nitric oxide levels. ATP is the main energy source for the majority of cellular functions, including biosynthetic reactions, motility, and cell division. Nitric oxide is an important biological messenger which can signal the smooth muscle surrounding a blood vessel to relax, thus resulting in vasodilation and increased blood flow. The phototherapy treatment can be applied to the patient at certain time periods before the transvenous lead placement as well as during the lead placement procedure. When applied at a relatively long time period (e.g. several months or weeks) before the lead placement, the phototherapy can help elevate cytoprotective heat shock proteins, improve vascular endothelial growth, and enhance angiogenesis (growth of new blood vessels). When applied at a short time period (e.g. a few days or hours) before or during the lead placement procedure, the phototherapy can help increase blood flow, reduce inflammation, and dilate the blood vessel. All these therapeutic effects facilitate the optimal positioning of the transvenous leads.

The preferred laser output wavelength for this exemplary embodiment is from 600 nm to 1500 nm, more preferably from 800 nm to 1000 nm, falling in the red to near infrared (NIR) range of the optical spectrum. The laser light in this wavelength range has been demonstrated to be beneficial for increasing cytochrome oxidase activity and ATP content, promoting wound healing, increasing blood flow, and reducing inflammation. In addition, it can penetrate deep (with a penetration depth greater than several centimeters) into the skin for treating internal organs and blood vessels. The output power of the laser is preferably above 0.5 watts to further increase the penetration depth of the laser light into the tissue. A cooling device can be used in combination with the high power laser to keep the surface temperature of the treatment area below a safety level. The cooling device can be a heat conductive material (such as sapphire) or a heat absorbent material (such as phase change material) that is transparent to the laser light or a cooling unit that delivers cold material (cold air, water, etc.) to extract heat from the treatment area. The laser light source may comprise multiple lasers with different output wavelengths, each wavelength matching with the absorption band of a specific chromophore (water, hemoglobin, lipid, protein, etc.) of the subject tissue and blood vessel. For example, the laser light source may comprise two high power diode lasers, one with an output wavelength of 810 nm, the other with an output wavelength of 980 nm. The 810 nm wavelength is well absorbed by the hemoglobin and melanin content of the biological tissue, while the 980 nm wavelength is efficiently absorbed by the water content. The outputs of the multiple laser sources can be combined at adjustable proportions and simultaneously applied to the subject tissue to achieve an enhanced treatment result. The laser sources may operate in a pulsed mode such that a high peak power is produced to increase the penetration depth of the laser light and/or to trigger nonlinear photochemical reactions yet the average power of the laser light is maintained at low levels to avoid any tissue damage.

To further increase the penetration depth of the laser light, the output wand 106 may comprise a protuberance (not shown) at the distal end, which is made of a material substantially transparent at the output wavelength of the laser light source. The protuberance is placed in close contact with the skin tissue of the treatment area. When a force is applied onto the output wand 106, the protuberance executes rubbing and kneading massage to the skin tissue. In the mean time, the laser light transmits through the protuberance to provide phototherapy to the treatment area. The massage action causes a reduction in skin thickness and an increase in skin density. This change in optical property of the skin helps to reduce the overall absorption and scattering loss of the skin and allows the laser light to penetrate deeper to treat internal organs and blood vessels. In addition, the mechanical massage causes an increase in blood circulation and fluid mobilization of the subcutaneous tissue, which enhances the therapeutic effects of the laser light. In the present embodiment, the protuberance is preferably made of sapphire, which offers excellent light transmission and good heat conductivity to reduce surface temperature of the subject treatment area.

In a slight variation of the present embodiment, the laser light source may be replaced with other types of light sources such as lamps, light emitting diodes (LEDs), superluminescent diodes (SLDs), etc. The therapeutic light beam can be directly delivered from the light source onto the subject treatment area without using any optical waveguides, thus avoiding the excessive light intensity loss caused by the inclusion of the optical waveguides. Besides the above disclosed applications, the laser therapy method may also aid in gaining venous access for angioplasty procedures, where balloons, stents, or other medical devices are employed to widen the diameter of blocked blood vessels in the heart, kidney, leg, and neck of the patient.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A method for placing a medical device into a blood vessel of a patient, the method comprising the steps of:
providing at least one light source to produce a therapeutic light beam;
non-invasively treating the patient with said therapeutic light beam from outside of the patient's body, said therapeutic light beam penetrating into the patient's body to dilate the blood vessel and increase a blood flow in the blood vessel; and
placing the medical device into the dilated blood vessel of the patient.

2. The method of claim 1, wherein the medical device comprises a cardiac resynchronization lead.

3. The method of claim 1, wherein the medical device comprises an angioplastic balloon and/or stent.

4. The method of claim 1, wherein the at least one light source has an output wavelength in the range of 600-1500 nm.

5. The method of claim 1, wherein the at least one light source has an output wavelength in the range of 800-1000 nm.

6. The method of claim 1, wherein the at least one light source comprises a laser.

7. The method of claim 6, wherein the laser has an output power of greater than 0.5 watts.

8. The method of claim 1, wherein the at least one light source comprises multiple lasers with different output wavelengths.

9. The method of claim 1, further comprising a step of providing a cooling device to keep a surface temperature of the patient's body below a safety level.

10. The method of claim 1, further comprising a step of applying a mechanical massage to skin tissue of the patient, wherein said mechanical massage causes a reduction in skin thickness and an increase in skin density to increase a penetration depth of said therapeutic light beam.

11. The method of claim 10, wherein said mechanical massage is applied through a protuberance substantially transparent to said therapeutic light beam.

12. The method of claim 1, further comprising a step of delivering said therapeutic light beam with an optical waveguide.
